(19) United States
(12) Patent Application Publication
(10) Pub. No.: US 2026/0355389 A1
(43) Pub. Date: Nov. 12, 2026

(54) DOUBLE-NETWORK SOFT TISSUE FILLING MEDICAL HYDROGEL, PREPARATION METHOD THEREFOR, AND USE THEREOF

(71) Applicant: SHANGHAI RUINING BIOTECHNOLOGY CO., LTD., Shanghai (CN)

(72) Inventors: Zhenkun YAN, Shanghai (CN); Zhanpeng ZHANG, Shanghai (CN); Zhen PAN, Shanghai (CN)

(21) Appl. No.: 19/716,559

(22) Filed: Jun. 23, 2026

Related U.S. Application Data
(63) Continuation of application No. PCT/CN2024/116862, filed on Sep. 4, 2024.

(30) Foreign Application Priority Data
Dec. 29, 2023 (CN) 202311863891.9

(51) Int. Cl. A61L 27/52; A61L 27/02; A61L 27/18

(52) U.S. Cl. CPC A61L 27/52; A61L 27/025; A61L 27/18; A61L 2430/34

(57) ABSTRACT

A double-network soft tissue filling medical hydrogel, a preparation method therefor, and a use thereof. The hydrogel comprises the following components: L-polyglutamic acid and polylysine, 1-ethyl-(3-dimethylaminopropyl)carbodiimide hydrochloride, N-hydroxysuccinimide, and calcium chloride dihydrate. The hydrogel has good and controllable mechanical properties, and has a controllable in-vivo degradation rate.

### CROSS-REFERENCE TO RELATED APPLICATIONS

This application is a continuation of International Patent Application No. PCT/CN2024/116862, filed on Sep. 4, 2024, which claims priority to Chinese Patent Application No. 202311863891.9, filed on Dec. 29, 2023. The entire contents thereof are incorporated herein by reference in their entirety.

### FIELD OF TECHNOLOGY

The present invention relates to the technical field of soft tissue filling medical hydrogels, particularly relates to the IPC C08G65 field, and more specifically relates to a double-network soft tissue filling medical hydrogel, a preparation method therefor, and use thereof.

### BACKGROUND

The most commonly used materials for preparing biomimetic soft tissue substitutes in domestic and international research are polymeric hydrogels. The polymeric hydrogels are composed of high polymers with network structures and water, and have strong water absorption, swelling property, thixotropy, dehydration shrinkage, elasticity, and biocompatibility. The advantage of injecting soft tissue filling materials is that clinical surgery is simple in operation, minimally invasive, and significant in therapeutic effect. However, the soft tissue filling materials currently used clinically still have many shortcomings. For example, microsphere-type soft tissue filling materials, such as polytetrafluoroethylene microspheres, silicone resin balls, and ceramic microspheres, are prone to undergoing migration, especially in soft tissues of lumina, resulting in the need for a secondary injection to enhance efficacy. Although autologous fat injection exhibits good biocompatibility, it is rapidly reabsorbed in vivo and has a short duration of efficacy. Although natural polymeric hydrogels, such as hyaluronic acid and collagen, have good biocompatibility, these materials are decomposed by enzymes in vivo, resulting in similarly short efficacy.

The foreign product Bulkamid has been approved in the United States of America for use as a soft tissue filling material for patients with urinary incontinence. Due to its mechanical properties being similar to those of soft tissues of the human body, it has a good clinical effect. However, its main ingredient, polyacrylamide, contains toxic monomers, purification processes for synthetic materials have high requirements, and adverse events caused by the problem of raw material purity have been widely reported, so that the product has a higher safety risk.

CN102093576B discloses a method for preparing a polyvinyl alcohol hydrogel. The method of the invention is simple in operation and easy to implement, and parameters can be adjusted according to characteristics of different tissues, making the hydrogel easy to obtain. A biomimetic soft tissue material prepared by the method exhibits stable properties, is easy to store, and can be recycled, but its mechanical properties are still not high enough.

Therefore, in view of the above situations, it is necessary to develop a soft tissue filling medical hydrogel that has good material safety, mechanical properties consistent with properties of soft tissues of the human body, and long-term stability in vivo.

### SUMMARY

A first aspect of the present invention provides a double-network soft tissue filling medical hydrogel, which includes the components of L-polyglutamic acid and polylysine, 1-ethyl-(3-dimethylaminopropyl)carbodiimide hydrochloride (EDC·HCl), N-hydroxysuccinimide (NHS), and calcium chloride dihydrate.

A second aspect of the present invention provides a method for preparing the double-network soft tissue filling medical hydrogel, which includes the following steps:
step 1. taking and dissolving L-polyglutamic acid in a morpholinoethanesulfonic acid solution to obtain a solution A;
step 2. taking and dissolving polylysine in a morpholinoethanesulfonic acid solution to obtain a solution B;
step 3. adding the above solution B dropwise to the solution A, and mixing uniformly to obtain a mixed system C for later use;
step 4. taking and dissolving 1-ethyl-(3-dimethylaminopropyl)carbodiimide hydrochloride in a morpholinoethanesulfonic acid solution to obtain a solution D;
step 5. taking and dissolving N-hydroxysuccinimide in a morpholinoethanesulfonic acid solution to obtain a solution E;
step 6. taking and dissolving calcium chloride dihydrate in deionized water to obtain a solution F; and
step 7. taking and adding the solutions D, E, and F dropwise to the system C, stirring, and then placing the mixture in pure water for dialysis to obtain the hydrogel.

A mass concentration percentage of the L-polyglutamic acid is 3-15%, preferably 10%.

A mass concentration percentage of the polylysine is 1-3%, preferably 2.5%.

A mass concentration percentage of the EDC·HCl and NHS is 0.04-0.24%, preferably 0.2%.

A mass concentration percentage of the calcium chloride dihydrate is 0-0.06%, preferably 0.048%.

A concentration of the morpholinoethanesulfonic acid solution is 0.05-0.5 mol/L.

A weight ratio of the L-polyglutamic acid to the polylysine is (4-10):1.

It is found by the applicant through research that when the weight ratio of the L-polyglutamic acid to the polylysine is (4-10):1, mechanical properties of the hydrogel can be effectively improved, which may be because the L-polyglutamic acid and the polylysine form a systemic polymer with a single-network structure having the characteristics of “a long chain and multiple arms” through bonding, and the polymer further chelates with a chelating agent to form a system with a double-network structure. When the content of the L-polyglutamic acid is too low, a precipitate easily occurs, and the hydrogel is not formed. It is further found through research that when a volume ratio of the solutions D, E, and F is (0.2-0.5):(0.2-0.5):(0.25-1), a swelling degree of the hydrogel can be further improved, and meanwhile, a degradation rate is regulated. This may be because-COOH in the polyglutamic acid is activated by the 1-ethyl-(3-dimethylaminopropyl)carbodiimide hydrochloride (EDC·HCl) and the N-hydroxysuccinimide (NHS) as activators, and then forms an amide bond with —NH₂ of the polylysine to constitute a first layer of cross-linked network. Subsequently, the calcium chloride forms a chelate bond with free-COOH in the polyglutamic acid to constitute a second layer of cross-linked network. Due to a combined effect of the two networks, overall performance of the hydrogel is effectively improved.

Preferably, the weight ratio of the L-polyglutamic acid to the polylysine is (4-8):1.

A volume ratio of the solutions D, E, and F is (0.2-0.5):(0.2-0.5):(0.25-1).

Preferably, the volume ratio of the solutions D, E, and F is 0.5:0.5:(0.5-1).

A solid content of the hydrogel is 15-30 wt %.

Preferably, the solid content of the hydrogel is 15-25 wt %.

A swelling degree of the hydrogel is ≥100%.

Preferably, the swelling degree of the hydrogel is ≥200%.

Further preferably, the swelling degree of the hydrogel is ≥300%.

A degradation time of the hydrogel is 3-20 days.

Preferably, the degradation time of the hydrogel is 7-12 days.

A third aspect of the present invention provides use of the method for preparing the double-network soft tissue filling medical hydrogel, wherein the hydrogel can be used as a soft tissue filling material in one of facial filling in medical aesthetics, stress urinary incontinence, fecal incontinence, vesicoureteral reflux, and esophageal reflux.

### Beneficial Effects

1. The weight ratio of the L-polyglutamic acid to the polylysine is (4-10):1, which can effectively improve the mechanical properties of the hydrogel.

2. The volume ratio of the solutions D, E, and F is (0.2-0.5):(0.2-0.5):(0.25-1), which can further improve the swelling degree of the hydrogel, and meanwhile, regulate the degradation rate.

3. The volume ratio of the solutions D, E, and F is 0.5:0.5:(0.5-1), and the swelling degree of the hydrogel is ≥300%.

4. The solid content of the hydrogel is 15-30 wt %, which facilitates the formation of the double-network hydrogel and can allow for injection filling, thereby meeting requirements for properties of hydrogels in the injectable biomedical field.

5. The hydrogel of the present invention has good and controllable mechanical properties and a degradation rate of 3-20 days, and can be used as a soft tissue filling material in the medical field.

### BRIEF DESCRIPTION OF THE DRAWINGS

FIG. 1 shows a hydrogel prepared in Example 4.

FIG. 2 shows an image of the hydrogel prepared in Example 4 being injected through a syringe.

FIG. 3 shows a viscoelasticity test diagram of a hydrogel prepared in Example 1.

FIG. 4 shows a viscoelasticity test diagram of the hydrogel prepared in Example 4.

FIG. 5 shows a viscoelasticity test diagram of a hydrogel prepared in Example 15.

### DETAILED DESCRIPTION OF THE EMBODIMENTS

The following provides a detailed description of embodiments of the present invention in conjunction with examples. However, those skilled in the art will understand that the following examples are only for illustrating the present invention and should not be construed as limiting the scope of the present invention. If specific conditions are not indicated in the examples, conventional conditions or those recommended by manufacturers are used. All reagents and instruments used without specific manufacturers can be purchased on the market.

### Examples 1-18: a Double-Network Soft Tissue Filling Medical Hydrogel.

### Comparative Example 1: 5% Hyaluronic Acid Solution.

### Raw Materials for Preparation are Shown in Table 1:

TABLE 1

| | | | | | | | Calcium |
|---|---|---|---|---|---|---|---|
| | L-polyglutamic acid | | Polylysine solution | | EDC•HCl | NHS | chloride |
| | solution (solution A) | | (solution B) | | solution/ml | solution/ml | solution/ml |
| Example | Concentration | Volume/ml | Concentration | Volume/ml | (solution D) | (solution E) | (solution F) |
| 1 | 10% | 10 | 2.50% | 10 | 0.5 | 0.5 | 0 |
| 2 | 10% | 10 | 2.50% | 10 | 0.5 | 0.5 | 0.25 |
| 3 | 10% | 10 | 2.50% | 10 | 0.5 | 0.5 | 0.5 |
| 4 | 10% | 10 | 2.50% | 10 | 0.5 | 0.5 | 1 |
| 5 | 10% | 10 | 1.00% | 10 | 0.5 | 0.5 | 0.5 |
| 6 | 10% | 10 | 5.00% | 10 | 0.5 | 0.5 | 0.5 |
| 7 | 2.50% | 10 | 2.50% | 10 | 0.5 | 0.5 | 0.5 |
| 8 | 10% | 10 | 2.50% | 10 | 0.5 | 1 | 0.5 |
| 9 | 10% | 10 | 2.50% | 10 | 0.5 | 2 | 0.5 |
| 10 | 10% | 10 | 2.50% | 10 | 0.2 | 0.5 | 0.5 |
| 11 | 10% | 10 | 2.50% | 10 | 1 | 0.5 | 0.5 |
| 12 | 5% | 10 | 2.50% | 10 | 0.5 | 0.5 | 0.5 |
| 13 | 5% | 10 | 1.25% | 10 | 0.25 | 0.25 | 0.25 |
| 14 | 10% | 9 | 2.50% | 9 | 0.45 | 0.45 | 0.45 |
| 15 | 10% | 15 | 2.50% | 10 | 0.5 | 0.5 | 0.5 |
| 16 | 10% | 9 | 2.50% | 9 | 0.45 | 0.45 | 0.45 |
| 17 | 9% | 10 | 2.25% | 10 | 0.5 | 1 | 0.5 |
| 18 | 10% | 10 | 2.50% | 12.5 | 0.5 | 0.5 | 0.5 |
| Comparative | 5% hyaluronic acid solution | | | | | | |
| Example 1 | | | | | | | |

The L-polyglutamic acid had a molecular weight of 700,000 and was provided by Shanghai Aladdin Biochemical Technology Co., Ltd.; the polylysine had a model of P192512 and a molecular weight of 2,000-5,000 and was provided by Shanghai Aladdin Biochemical Technology Co., Ltd.; and the EDC·HCl and NHS were provided by Shanghai Aladdin Biochemical Technology Co., Ltd.

In Example 1, a method for preparing the double-network soft tissue filling medical hydrogel includes the following steps:
step 1: taking and dissolving the L-polyglutamic acid in a morpholinoethanesulfonic acid (MES) solution to obtain a solution A for later use, wherein a mass fraction of the L-polyglutamic acid in the solution A is shown in Table 1;
step 2: taking and dissolving the polylysine in an MES solution to obtain a solution B for later use, wherein a mass fraction of the polylysine in the solution B is shown in Table 1;
step 3: adding the above solution B dropwise to the solution A, and mixing uniformly to obtain a mixed system C for later use;
step 4: taking and dissolving 0.5 g of EDC·HCl in 2 g of an MES solution to obtain a solution D for later use;
step 5: taking and dissolving 0.5 g of NHS in 2 g of an MES solution to obtain a solution E for later use; and
step 6: separately taking and adding the solutions D and E dropwise to the system C, stirring for 5 minutes, and then placing the mixture in pure water for dialysis at room temperature for 24 hours to obtain the hydrogel.

The morpholinoethanesulfonic acid solution had a concentration of 0.1 mol/L, with water as a solvent.

In Examples 2-5 and 8-17, a method for preparing the double-network soft tissue filling medical hydrogel includes the following steps:
step 1: taking and dissolving the L-polyglutamic acid in a morpholinoethanesulfonic acid (MES) solution to obtain a solution A for later use, wherein a mass fraction of the L-polyglutamic acid in the solution A is shown in Table 1;
step 2: taking and dissolving the polylysine in an MES solution to obtain a solution B for later use, wherein a mass fraction of the polylysine in the solution B is shown in Table 1;
step 3: adding the above solution B dropwise to the solution A, and mixing uniformly to obtain a mixed system C for later use;
step 4: taking and dissolving 0.5 g of EDC·HCl in 2 g of an MES solution to obtain a solution D for later use;
step 5: taking and dissolving 0.5 g of NHS in 2 g of an MES solution to obtain a solution E for later use;
step 6: taking and dissolving 0.56 g of CaCl₂·2H₂O in 10 mL of deionized water to obtain a solution F; and
step 7: separately taking and adding the solutions D, E, and F dropwise to the system C, stirring for 5 minutes, and then placing the mixture in pure water for dialysis at room temperature for 24 hours to obtain the hydrogel.

The morpholinoethanesulfonic acid solution had a concentration of 0.1 mol/L, with water as a solvent.

The CaCl₂·2H₂O was provided by Shanghai Aladdin Biochemical Technology Co., Ltd.

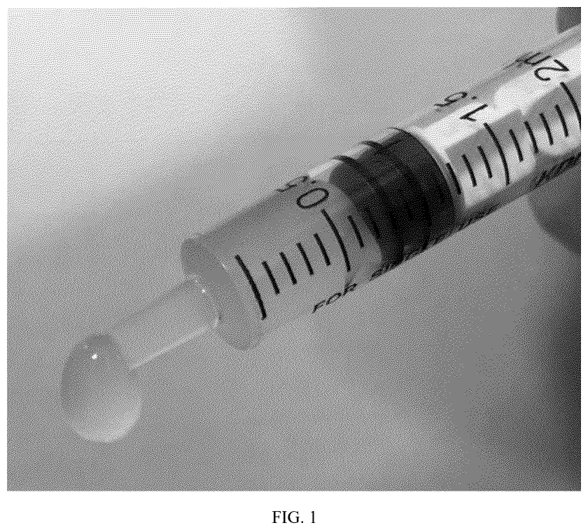

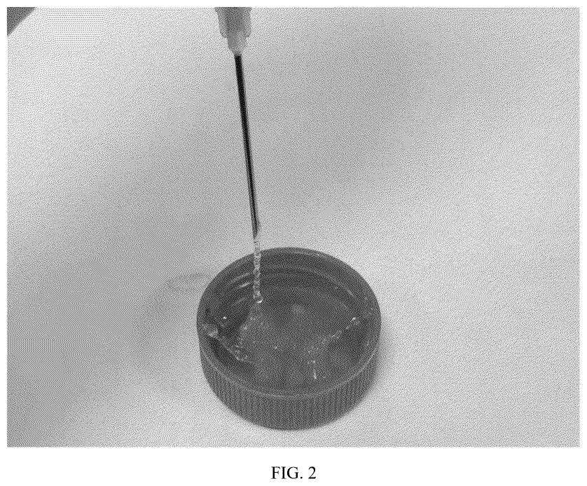

The hydrogel prepared in Example 4 is shown in FIG. 1, which can be injected through a syringe (FIG. 2).

In Examples 6, 7, and 18, a method for preparing the double-network soft tissue filling medical hydrogel includes the following steps:
step 1: taking and dissolving the L-polyglutamic acid in a morpholinoethanesulfonic acid (MES) solution to obtain a solution A for later use, wherein a mass fraction of the L-polyglutamic acid in the solution A is shown in Table 1;
step 2: taking and dissolving the polylysine in an MES solution to obtain a solution B for later use, wherein a mass fraction of the polylysine in the solution B was shown in Table 1; and
step 3: adding the above solution B dropwise to the solution A, and mixing uniformly to obtain a mixed system C until a precipitate occurs.

In Comparative Example 1, a method for preparing a hydrogel includes: adding 0.5 g of hyaluronic acid to 9.95 g of water under stirring to obtain a gel medium.

### Performance Test Methods

The following tests were conducted on the hydrogels of Examples 1-18 and Comparative Example 1. Test results are shown in Table 2.

### 1. Swelling Ratio

Specific implementation method: A PBS solution at 10 times the mass was added into a reagent flask loaded with a gel system, followed by swelling at 37 °C for 24 h. A formula of the swelling ratio is:

swelling ratio=(mass after swelling−mass before swelling)/mass before swelling*100%.

### 2. Degradation Rate

Specific implementation method: A PBS solution at 10 times the mass was added into a reagent flask loaded with a gel system, followed by regular observation at 37 °C, and the mass of the gel was recorded, wherein “−” indicated that a degradation test was not conducted.

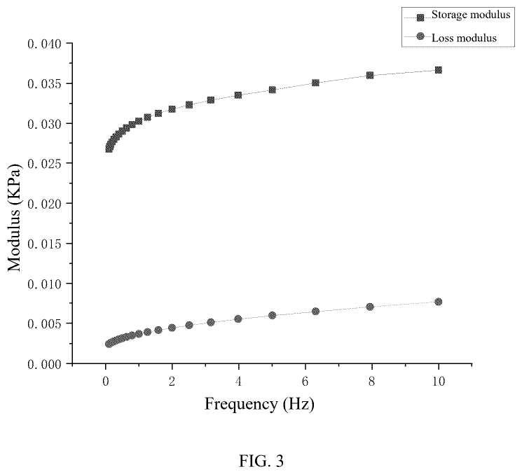

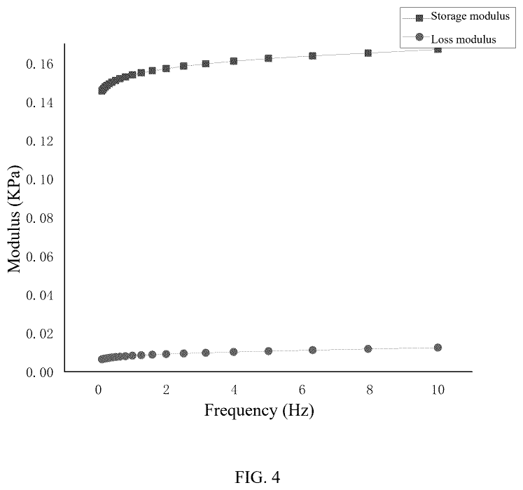

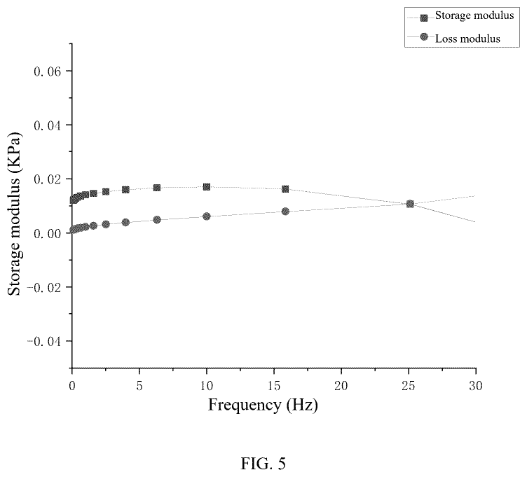

### 3. a Viscoelasticity Test was Conducted on Examples 1, 4, and 15. Test Diagrams are Shown in FIGS. 3-5.

Specific implementation method: Samples were subjected to constant-temperature treatment at 37 °C and then placed in a rotational rheometer to test the storage modulus and loss modulus of the samples at 0.1-1,000 Hz.

### Performance Test Data

TABLE 2

| | | Complete | |
|---|---|---|---|
| | | degradation | |
| Example | Swelling degree | time | |
| 1 | 139.55% | 12 | days |
| 2 | 240.05% | 9 | days |
| 3 | 208.96% | 9 | days |
| 4 | 311.65% | 12 | days |
| 5 | 134.87% | 14 | days |
| 6 | No gel formation (precipitate) | — | |
| 7 | No gel formation (precipitate) | — | |
| 8 | 136.77% | 12 | days |
| 9 | 146.64% | 6 | days |
| 10 | No gel formation (liquid) | — | |
| 11 | 84.93% | 12 | days |
| 12 | No gel formation (liquid) | — | |
| 13 | No gel formation (liquid) | — | |
| 14 | No gel formation (viscous liquid) | — | |
| 15 | 186.19% | 9 | days |
| 16 | Gel fragmentation | 3 | days |
| 17 | No gel formation (liquid) | — | |
| 18 | No gel formation (precipitate) | — | |
| Comparative | −18.71% | 7 | days |
| Example 1 | | | |

Compared with Examples 6, 7, 12, and 18 in which aggregation and precipitation of polyamino acids occurred during mixing of the polyglutamic acid and the polylysine, it can be concluded that in the double-network hydrogel system described herein, the content of the polyglutamic acid cannot be less than 4 times that of the polylysine.

Compared with Examples 14, 16, and 3, it can be considered that the substance contents of Examples 14 and 16 are 80% and 90% of that of Example 3, respectively. According to curing conditions of both, it can be considered that in a double-network viscoelastic hydrogel, its solid content cannot be lower than that in Example 16.

What is claimed is:

1. A double-network soft tissue filling medical hydrogel, comprising the components of L-polyglutamic acid and polylysine, 1-ethyl-(3-dimethylaminopropyl)carbodiimide hydrochloride, N-hydroxysuccinimide, and calcium chloride dihydrate.

2. A method for preparing the double-network soft tissue filling medical hydrogel according to claim 1, comprising the following steps:
step 1: taking and dissolving L-polyglutamic acid in a morpholinoethanesulfonic acid solution to obtain a solution A;
step 2: taking and dissolving polylysine in a morpholinoethanesulfonic acid solution to obtain a solution B;
step 3: adding the above solution B dropwise to the solution A, and mixing uniformly to obtain a mixed system C;
step 4: taking and dissolving 1-ethyl-(3-dimethylaminopropyl)carbodiimide hydrochloride in a morpholinoethanesulfonic acid solution to obtain a solution D;
step 5: taking and dissolving N-hydroxysuccinimide in a morpholinoethanesulfonic acid solution to obtain a solution E;
step 6: taking and dissolving calcium chloride dihydrate in deionized water to obtain a solution F; and
step 7: taking and adding the solutions D, E, and F dropwise to the mixed system C, stirring, and then placing the mixture in pure water for dialysis to obtain the hydrogel.

3. The method for preparing the double-network soft tissue filling medical hydrogel according to claim 2, wherein a concentration of the morpholinoethanesulfonic acid solution is 0.05-0.5 mol/L.

4. The method for preparing the double-network soft tissue filling medical hydrogel according to claim 3, wherein a weight ratio of the L-polyglutamic acid to the polylysine is (4-10):1.

5. The method for preparing the double-network soft tissue filling medical hydrogel according to claim 4, wherein the weight ratio of the L-polyglutamic acid to the polylysine is (4-8):1.

6. The method for preparing the double-network soft tissue filling medical hydrogel according to claim 5, wherein a volume ratio of the solutions D, E, and F is (0.2-0.5):(0.2-0.5):(0.25-1).

7. The method for preparing the double-network soft tissue filling medical hydrogel according to claim 6, wherein the volume ratio of the solutions D, E, and F is 0.5:0.5:(0.5-1).

8. The method for preparing the double-network soft tissue filling medical hydrogel according to claim 7, wherein a swelling degree of the hydrogel is ≥100%.

9. The method for preparing the double-network soft tissue filling medical hydrogel according to claim 8, wherein a degradation time of the hydrogel is 3-20 days.

10. Use of the method for preparing the double-network soft tissue filling medical hydrogel according to claim 2, wherein the hydrogel is used as a soft tissue filling material in one of facial filling in medical aesthetics, stress urinary incontinence, fecal incontinence, vesicoureteral reflux, and esophageal reflux.
